(19) United States
(12) Patent Application Publication
(10) Pub. No.: US 2021/0212669 A1
(43) Pub. Date: Jul. 15, 2021

(54) SLEEVE

(71) Applicant: TSC, LLC, Holladay, UT (US)

(72) Inventors: Brian Pratt, Holladay, UT (US); Conor Pratt, Holladay, UT (US)

(21) Appl. No.: 17/150,090

(22) Filed: Jan. 15, 2021

Related U.S. Application Data
(60) Provisional application No. 62/961,534, filed on Jan. 15, 2020.

(51) Int. Cl. A61B 10/00; A61F 5/453

(52) U.S. Cl. CPC A61B 10/0058; A61F 5/453

(57) ABSTRACT

A modular system which may aid in collection of bodily fluids, and more particularly collection of sperm. The system may include a plurality of components including a sleeve, a core and at least one cap. The system may be easily assembled and disassembled for interchangeability of each of its components as well as for cleaning and disinfection. The system may include a substantially rigid outer shell with a more malleable inner core. The core may be a single piece or multiple pieces and may be interchangeable depending on a user's preference and may reside within the outer shell but may be easily removable from the outer shell. A first end cap may include a face of substantially the same material as the inner core and may be interchangeable based on user preference as well.

### PRIORITY CLAIM

This application claims priority to U.S. Provisional Application 62/961,534, filed Jan. 15, 2020, entitled SLEEVE (“the '534 Provisional application”), is hereby made pursuant to 35 U.S.C. § 119(e). The entire disclosures of the '503 Provisional application is are hereby incorporated herein.

### TECHNICAL FIELD

This disclosure relates generally to a silicone sleeve that may aid in collection of bodily fluids, and more particularly collection of sperm. The sleeve may be modular to allow for easy manipulation and cleaning of the apparatus to prevent bacterial or other biological, or microbiological, buildup which may lead to infection. The sleeve may be easily assembled and disassembled for continuous clean use.

### RELATED ART

Sperm collecting apparatuses have been utilized for decades for providing an easy means of collecting human bodily fluids. These apparatuses have been provided for medical purposes, medical research and medical treatments. These apparatuses have also been utilized for recreation to provide a means for human male ejaculation. Often these apparatuses mimic genitalia, and more often female genitalia. These apparatuses are intended to provide friction for a user which may simulate sexual intercourse.

Primarily devices for collecting of sperm are fabricated from polymers. These polymers are typically a very pliable and forgiving rubber such as thermoplastic elastomer gel (TPE) or rubber. The outside of these devices is typically a more rigid material, which may also be a polymer. However, many of these devices utilize a single piece of polymer with a single type of rigidity both internally and externally.

The devices for sperm collection lack the ability to adequately clean and disinfect the devices. Many lack the ability to access the inside of device to clean out the collection apparatus after ejaculation has occurred. Many apparatuses include a single insertion point or access point with no access to the opposing end, or distal end of the device.

A device that is modular which allows for easy access to the internal structure of the sperm collection apparatus would be advantageous to allow for easy cleaning and disinfection as well as add to the longevity of the device.

### SUMMARY

A modular system may comprise a silicone sleeve of a first rigidity, the silicone sleeve may further comprise a cylindrical body and outer wall and an inner wall, a proximal end with a first circumferential ring. The cylindrical body may include a first shell portion and second shell portion which may be equal and mirrored halves. The system may also include a silicone core of a second rigidity different from the first rigidity and may also be mirrored halves. The modular system may also include a first cap positioned toward the proximal end, the first cap comprising a first aperture. The second end may be integral and/or molded with the cylindrical body.

The silicone core may be less rigid than the silicone sleeve. The silicone sleeve may comprise a first opening and a second opening. The silicone core comprises a first core opening and a second core opening.

Additionally, the silicone core may be positioned within the silicone sleeve and may comprise, a core proximal end with a core first ring. The silicone core may extend beyond the proximal end.

The first cap may engage both the core first ring and first circumferential ring. The first cap may include a face with the same rigidity of silicone as the silicone core. The first cap may also include an aperture resembling human genitalia.

A method of assembling and disassembling a collection system may comprise, providing a silicone sleeve with a first rigidity wherein the silicone sleeve may comprise a cylindrical body and outer wall and an inner wall, wherein the cylindrical body comprises to mirrored halves that engage to form the entire cylindrical body. A proximal end of the cylindrical body may include a first circumferential ring. The method may also include laying a silicone core with a second rigidity at least partially within at least one half of one of the first halves of the cylindrical body. A core proximal end may extend over the first circumferential ring and engage with a first cap.

The method of assembly may further include positioning the first cap over the core proximal end and first circumferential ring. The method may further include engaging the other half of the cylindrical body with the previous half to maintaining the silicone core within the cylindrical body of the silicone sleeve.

The method may also include snapping the first cap onto the silicone sleeve.

### BRIEF DESCRIPTION OF THE DRAWINGS

In the drawings:

FIG. 1 is a perspective front view of a sleeve fully assembled;

FIG. 2 is perspective back view of the sleeve of FIG. 1 fully assembled;

FIG. 3 is a side view of the sleeve of FIG. 1 fully assembled;

FIG. 4 is a front view of the sleeve of FIG. 1;

FIG. 5 is a back view of the sleeve of FIG. 1;

FIG. 6 is a perspective view of a first half of a shell of the sleeve of FIG. 1;

FIG. 7a is a back view of the shell of FIG. 6;

FIG. 7b is a perspective back view of the shell of FIG. 6;

FIG. 7c is a perspective side view of the shell of FIG. 6;

FIG. 7d is a perspective front view of the shell of FIG. 6.

FIG. 8 is a front view of the shell of FIG. 6;

FIG. 9 is a partial close up of front perspective view of the shell of FIG. 6 with a male and female portions of a fastening mechanism;

FIG. 10 is a partial close up of a back perspective view of the shell of FIG. 9;

FIG. 11 is a perspective back view of a partially disassembled (or cross-sectional) sleeve of FIG. 1 with a first half of a core;

FIG. 12 is a perspective view of a partially disassembled (or cross-sectional) sleeve of FIG. 1 with a first half of a core;

an exploded perspective view of the component parts of the sleeve of FIG. 1 including a first end cap, a second end cap, a first half of a core, a second half of the core, and an outer shell;

FIG. 13 is a cross-sectional side view of an alternate embodiment of the sleeve of FIG. 1;

FIG. 14a is a longitudinal side view of the outer shell of FIG. 13;

FIG. 14b is a perspective view of the outer shell of FIG. 13;

FIG. 15a is a perspective view of an alternate embodiment of core of FIG. 11;

FIG. 15b is a longitudinal side view of the core of FIG. 15a;

FIG. 15c is a front axial view of the core of FIG. 15a;

FIG. 15d is a cross-sectional, or partially disassembled half, longitudinal side view of the core of FIG. 15a;

FIG. 16a is a front perspective view of a first end cap of FIG. 1 or 13;

FIG. 16b is a back perspective view of the first end cap of FIG. 1 or 13;

FIG. 17a is a front perspective view of the second end cap of FIG. 13; and

FIG. 17b is a back perspective view of the second end cap of FIG. 13.

### DETAILED DESCRIPTION

The following description sets forth an embodiment of a system which may be a sleeve, or sperm collection sleeve, or modular sleeve, to be used as a collection mechanism for bodily fluids and namely sperm. The sleeve may be modular and allow for easy assembly and disassembly to allow access into the central portion of the sleeve for cleaning, disinfection or replacement of a core.

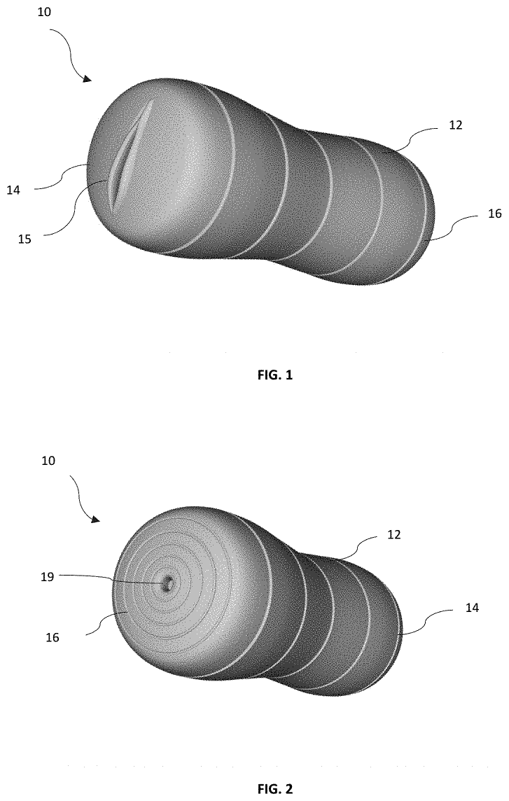

Referring to FIGS. 1-5, a system 10, or sleeve, or collection apparatus, or collection device, with a housing 12, or shell, a first cap 14, or front cap, and a second end 16, or end cap. The system 10 may include a central axis and be substantially cylindrical, or tubular in shape. The system may allow for engagement of the housing 12 with the first cap 14 toward a proximal end of the housing 12. The second end 16 may be integral and part of outer shell 12 and may be molded into a single piece, or multiple pieces as set forth herein.

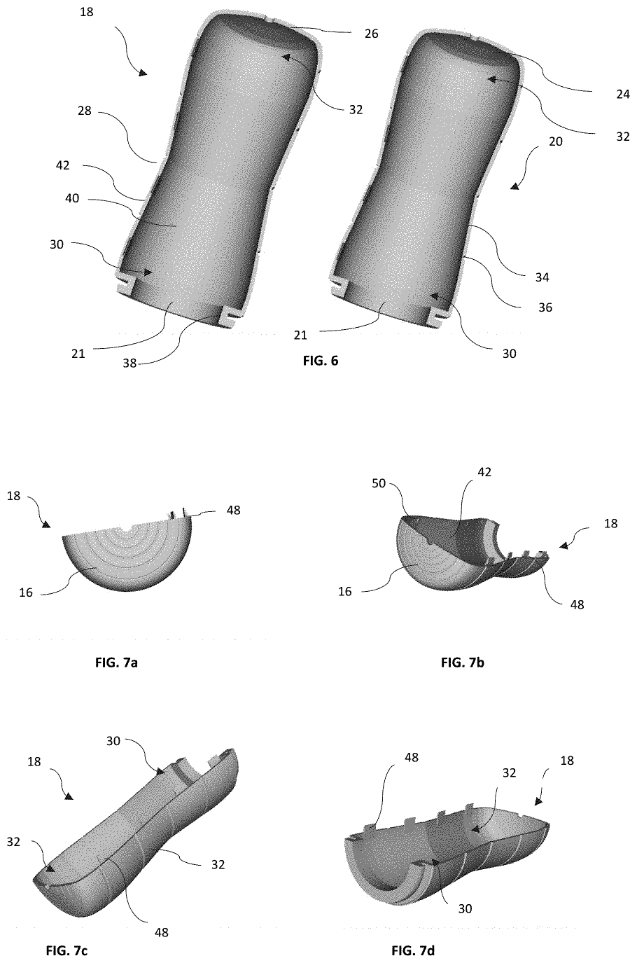

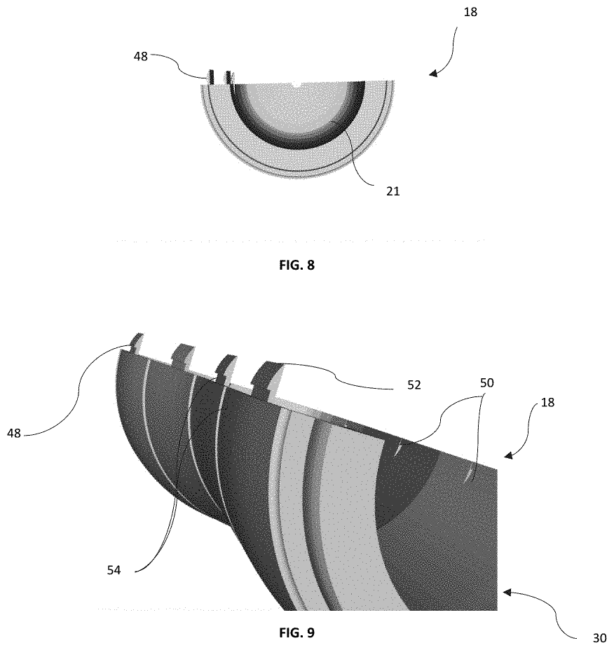

Referring to FIG. 6, the housing 12 may include a first shell portion 18, or first half shell, and a second shell portion 20, or second half. The first shell portion 18 and second shell portion 20 may comprise two halves that are similar, if not identical, in manufacture and interchangeable. The first shell portion 18 and second shell portion 20 may engage to form the shell 12. The second end 16 may include a first end portion 22 and a second end portion 24 that allows for flush engagement along a plane 26 defined by the two shell portions engaging each other. The first end portion 22 and second end portion 24 may each by semi-circular in shape to form a completed circular portion when engaged along the plane 26.

Referring to FIGS. 1-6, the housing 12 may be a rigid polymer or plastic and may provide a rigid support for the internal pieces of the system 10. The housing 12 may be comprises of shore A30 silicone, which may provide more rigidity than traditional silicone. However, it will be appreciated that a number of silicones and polymers are contemplated herein that may provide the same or similar rigidity. The housing 12 may be single injection molded halves of the first shell portion 18 and second shell portion 20. Alternatively, the housing 12 may be a single injection molded piece. Additionally, it may also be machined and may comprise two or more pieces as well. The housing 12 may comprise a longitudinal body 28 extending from a housing proximal end 30 to a housing distal end 32. The housing may include a circumferential outer wall 34 which may include ridges 36 or notches placed randomly, or equidistant from each other, along the length of the body 22. The housing 12 may be concave toward a center of the body 28 or taper toward a center of the body 28 to provide, in conjunction with the ridges 36, for easier handling and grip of the system 10.

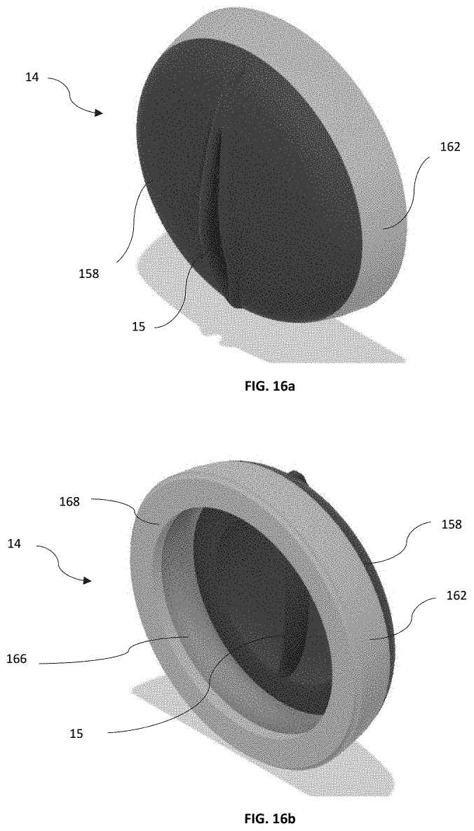

Referring to FIG. 1 and FIG. 4, a cap opening 15 is positioned along a central axis of the system 10. The cap opening 15 may be an elongated opening resembling female anatomy (See FIGS. 16a and 16b); however, it will be appreciated that any opening is contemplated such as circular, ovular, or any other polygonal shape.

Referring to FIG. 5, a hole 19, or aperture, may be positioned central to the second end 16. The hole 19 may be along the central axis of the system 10 and allow for air flow through the second end.

Referring to FIG. 6 each illustration may be either the first shell portion 18 or second shell portion 20, for ease in description this portion of the disclosure will refer to the first shell portion 18; however, each the of the first shell portion 18 and second shell portion 20 is interchangeable. Furthermore, and for purposes of this portion of the disclosure we will discuss the housing 12 as though a single unit and not as two halves which may allow FIG. 6 to be a cross sectional view.

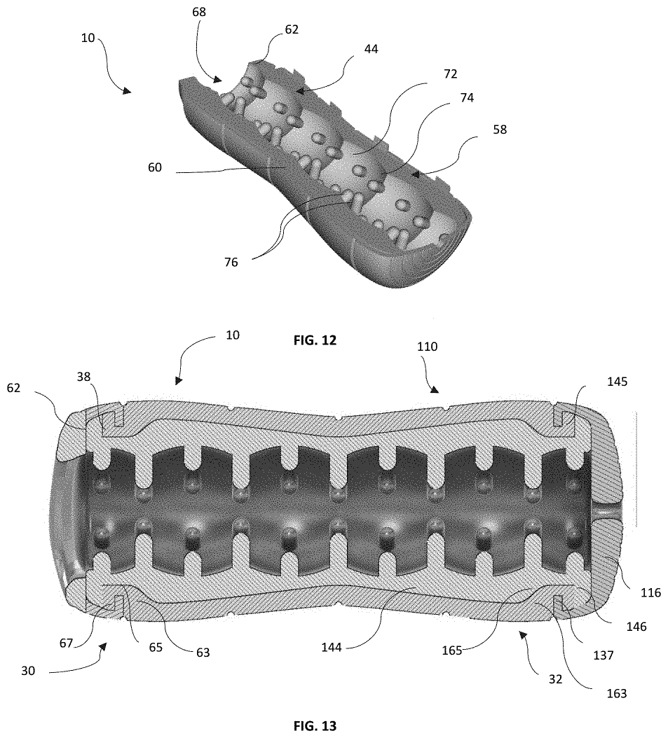

The housing proximal end 30 may include a first circumferential ring 38 configured to engage the first cap 14. The housing 12 may include a housing channel 40 extending longitudinally at least partially through the housing 12, and perhaps entirely through the housing 12, along the central axis. An inner wall 42 may extend circumferentially within the body 28 of the housing and have be substantially the same shape as the body 28, or outer body, of the housing 12, with the same or similar taper toward a center of the body 22. The inner wall 42 may be substantially smooth. The housing 12 may frictionally engage a core 44 (See FIG. 11 and FIG. 12) when the core 44 is positioned within the housing 12. Alternatively, the core 44 may be unremovably secured within the housing 12.

A shell opening 21 may be positioned toward the housing proximal end 30 and allow for passage of male anatomy into the inside of the housing 12 and ultimately the core 44.

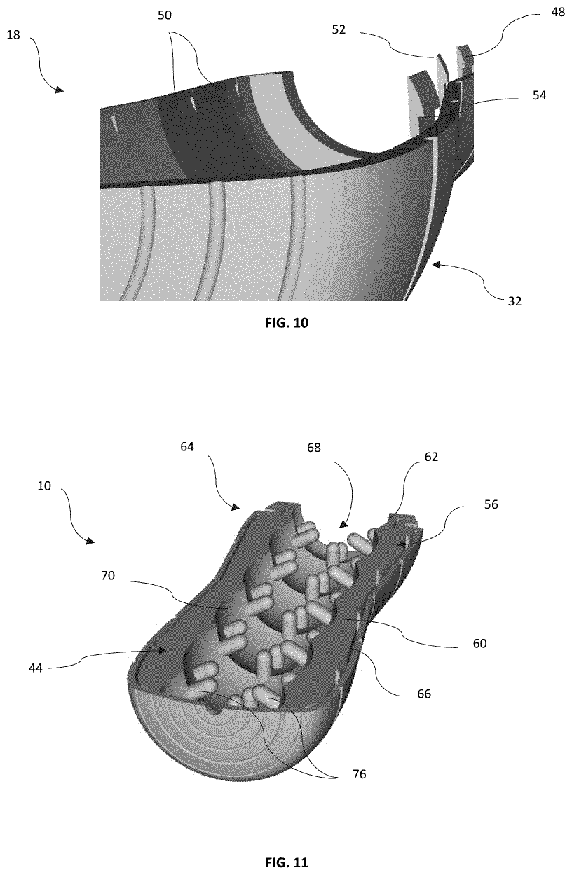

Referring to FIG. 7a thru FIG. 10 each illustration may be either the first shell portion 18 or second shell portion 20, for ease in description this portion of the disclosure will refer to the first shell portion 18; however, each the of the first shell portion 18 and second shell portion 20 is interchangeable. The first shell portion 18 may include at least one fastening mechanism 46, or latching mechanism, configured to engage the second shell portion 20, to form a single rigid housing 12. The fastening mechanism may include a protruding portion 48, or male portion, and a recessed portion 50, or female portion. The fastening mechanism 46 may include a plurality of fastening mechanisms. A plurality of protruding portions 46 may extend from the inner wall 42 of the housing 12 and extend beyond the plane 26 wherein the first shell portion 18 and second shell portion engage. The plurality of protruding portions 48 may be positioned equidistant along the longitudinal body 28 of the housing 12. While a single fastening mechanism 46 is sufficient multiple, or a plurality, of fastening mechanisms may provide greater securing of the first shell portion 18 and second shell portion to each other. Further, a plurality of fastening mechanisms may allow for easier assembly and disassembly of the system 10 to allow for ease of cleaning.

Opposite the protruding portion(s) 48 are the recessed portion(s) 50 configured to receive the protruding portions of the opposite shell portion (i.e. the recessed portion 50 of the first shell portion 18 receives the protruding portion 48 of the second shell portion 20). The protruding portion(s) 48 may include a pointed tip 52 toward the distal end of the protruding portion and a flange 54, or lip, shoulder, or ledge, that extends to form a clip that fits within the recessed portion(s) 50 to prevent removal of the two shell portions from one another unless a force is applied to disengage the flange 54 from the recessed portion 50. Alternatively, the recessed portion 50 may include an aperture that receives the protruding portion 48 with the flange 54 engaging a portion of that aperture. When a first shell portion 18 and second shell portion 20 are engaged then the inner wall 40 and outer wall 34 are flush.

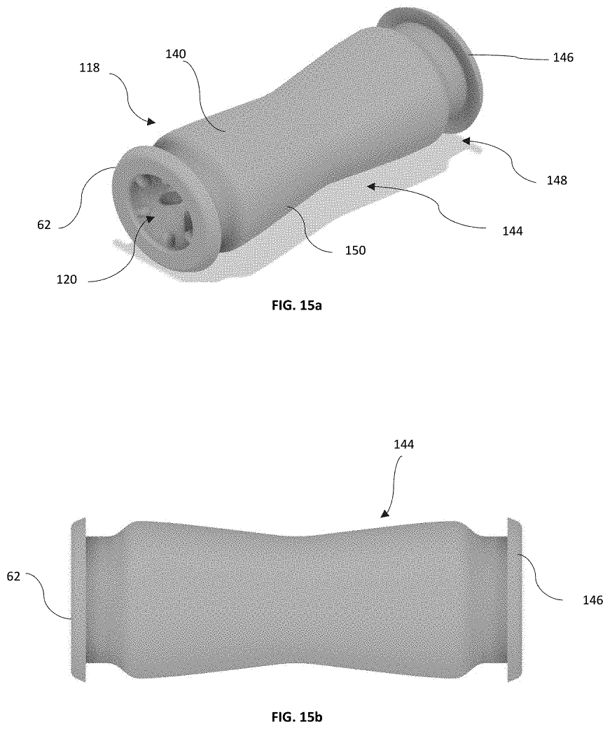

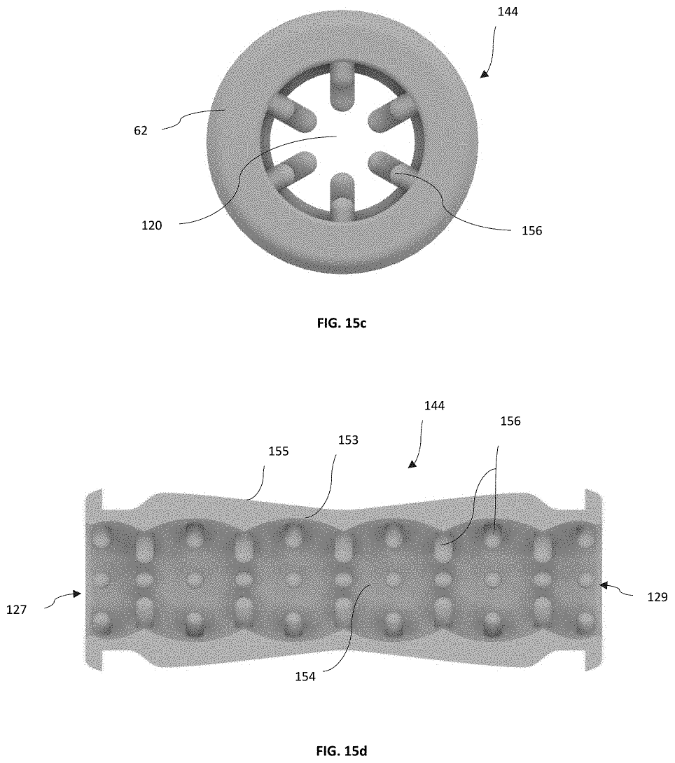

Referring to FIG. 11 and FIG. 12, the core 44 may be a single piece of material or may comprise a first core piece 56 (FIG. 11) and second core piece 58 (FIG. 12). Each of the first core piece 56 and second core piece 58 may be similar or identical and may be interchangeable. The core 44 may comprise a core body 60 which may be the same or similar in shape as the housing body 28 with a taper toward a center of the core 18. The core 18 may frictionally fit within the housing channel 40 providing for a complementary fit of the core 44 with the housing 12. The core 44 may include a core first ring 62 (more easily viewable in the alternate embodiment of FIGS. 15a and 15b) which may extend from a core proximal end 64 that corresponds with the housing proximal end 30. A distal end 65 of the core 44 may be rounded and may sit interiorly flush against the housing distal end 32.

The core 44 may include a circumferential core outer wall 66 which may be substantially smooth and engage the inner wall 40 of the housing 12. However, the outer wall 66 may be porous or rigid to provide greater friction. A core channel 68 may extend at least partially, and perhaps entirely, through a longitudinal length of the core 44 along the central axis of the sleeve 10. A core inner wall 70 may extend circumferentially through the core channel 68. The inner wall 70 may be waved, or comprise a plurality of valleys 72 and peaks 74 that extend longitudinally along the length of the core channel 68. A plurality of nubs 76, or ribs, or pots, or ribbing, extend from the core inner wall 70 toward the central axis and may be spaced in a pattern, or equidistant, from each other to provide a uniform “ribbing.” Alternatively, the nubs 76 may be positioned randomly along the core inner wall 70. The nubs 76 may provide feedback to a user of the system 10 and the friction which may simulate copulation. The number of nubs 76 may vary from 40-80 nubs and may provide sufficient friction with 66 nubs. The nubs may all be the same length or vary in length and be positioned within the core 44, extending from the core inner wall 70, in a pattern dependent on each length of the nubs 76. The nubs 76 may vary in length from 6 mm to 15 mm. The valleys 72 and peaks 74 may have nubs 76 extending from each and the longer nubs 76 may extend from the peaks 74 and the shorter nubs 76 from the valleys 72 or vice versa. The waved core inner wall 70 may provide for friction for the user as well.

The first core piece 56 and second core piece 58 may reside within the first shell portion 18 and second shell portion 20 respectively. However, it will be appreciated that each “half” is interchangeable with the other. The core 44 may be comprised of two, mirrored pieces of shore 00-30 silicone; however, it will be appreciated that different variations of silicone and polymers are contemplated.

Referring to FIG. 13, while this particular illustration references an alternate embodiment the proximal end 30 is substantially similar to the previous embodiment and for purposes of this disclosure is referenced herein with relation to the previous embodiment. The core first ring 62 may extend beyond the first circumferential ring 38 of the housing 12 exposing the core first ring 62 to the outside of the housing 12. A first shoulder 63 of the first circumferential ring 38 may reside within a first groove 65 of the core first ring 62 helping to retain and stretch the core 44 within the housing 12.

Referring to FIG. 13, with regard to an alternate embodiment, a system 110 is similar to system 10 however with the distal end 32 substantially similar to the proximal end 30. A core 144, similarly with a core second ring 146 may extend beyond the second circumferential ring 145 of the housing 12 exposing the core second ring 146 to the outside of the housing 12. A second shoulder 163 of the second circumferential ring 145 may reside within a second groove 165 of the core second ring 146 helping to retain and stretch the core 144 within the housing 12.

The system 10 is intended to be modular and allow a user to easily remove the first cap 14 the first shell portion 18 and second shell portion 20 as well as the core 44 (which may be a single piece or multiple pieces). Each of these elements may be interchangeable or replaced with different designs for the core 44 as well as the first cap 14. The first cap 14 may be provided with different sizes of apertures and different aesthetics to the appearance of genitalia. The core 44 may be changed to multiple core designs with different nubs, protrusions, waves, ridges or the like to provide different friction and feedback for a user. The modularity of the system 10 also provides for easy replacement of any component.

The system 10 may be easily manipulated and cleaned by removing the first cap 14 and the first shell portion 18 and second shell portion. The core 44 may then be easily removed with the open, or disengaged, configuration. The core 44, housing 12, first end cap 14 and first and second shell portions (18, 20), may be easily cleaned, allowed to dry and then replacing each part as indicated herein.

The system 10 may be manufactured with 100% medical grade platinum-cure silicone which is safe for human skin contact and highly resilient to extreme temperatures, chemicals and mechanical breakdown. Each element of the system 10 may be textured, even subtly textured, to provide extra grip and reduce the probability of lint collection.

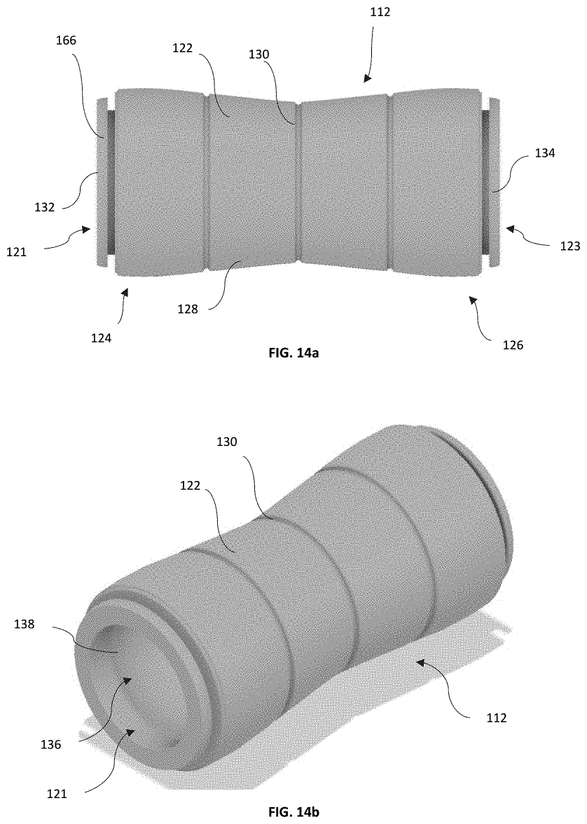

Referring to FIG. 14a and FIG. 14b, an alternate embodiment housing 112 may be a rigid polymer or plastic and may provide a rigid support for the internal pieces of the system 110. The housing 112 may be comprises of shore A30 silicone, which may provide more rigidity than traditional silicone. However, it will be appreciated that a number of silicones and polymers are contemplated herein that may provide the same or similar rigidity. The housing 112 may be a single injection molded piece; however, it may also be machined and may comprise two or more pieces as well. The housing 112 may comprise a longitudinal body 122 extending from a housing proximal end 124 to a housing distal end 126. The housing may include an outer wall 128 which may include ridges 130 or notches placed randomly, or equidistant from each other, along the length of the body 122. The housing 12 may be concave toward a center of the body 22 or taper toward a center of the body 212 to provide, in conjunction with the ridges 130, for easier handling and grip of the system 10.

The housing proximal end 124 may include a first circumferential ring 132 configured to engage the first cap 14. The housing distal end 126 may include a second circumferential ring 134 configured to engage a second cap 116. The housing 112 may include a housing channel 136 extending longitudinally at least partially through the housing 112, and perhaps entirely through the housing 112, along the central axis. An inner wall 138 may extend circumferentially within the body 22 of the housing and have be substantially the same shape as the body 122, or outer body, of the housing 112, with the same or similar taper toward a center of the body 122. The inner wall 138 may be substantially smooth. The housing 112 may frictionally engage the core 144 when the core is positioned within the housing 112.

Referring to FIGS. 15a-d, the core 144 may be a single piece of material or may comprise a first core piece and second core piece as set forth in the previous embodiment. The core 144 may comprise a core body 140 which may be the same or similar in shape as the housing body 122 with a taper toward a center of the core 144. The core 144 may frictionally fit within the housing channel 136 providing for a complementary fit of the core 144 with the housing 112. The core 144 may include the core first ring 62 which may extend from a core proximal end 118 that corresponds with the housing proximal end 124 and a core second ring 146 which may extend from a core distal end 148 which corresponds with the housing distal end 126.

The core 144 may include a circumferential core outer wall 150 which may be substantially smooth and engage the inner wall 138 of the housing 112. A core channel 120 may extend at least partially, and perhaps entirely, through a longitudinal length of the core 144 along the central axis of the sleeve 110. A core inner wall 154 may extend circumferentially through the core channel 120. The inner wall 54 may be waved, or comprise a plurality of valleys 153 and peaks 155 that extend longitudinally along the length of the core channel 120. A plurality of nubs 156, or ribs, or pots, or ribbing, extend from the core inner wall 154 toward the central axis and may be spaced in a pattern, or equidistant, from each other to provide a uniform “ribbing.” Alternatively, the nubs 156 may be positioned randomly along the core inner wall 154. The nubs 156 may provide feedback to a user of the system 110 and the friction which may simulate copulation. The number of nubs 156 may vary from 40-80 nubs and may provide sufficient friction with 156 nubs. The nubs may all be the same length or vary in length and be positioned within the core 144, extending from the core inner wall 154, in a pattern dependent on each length of the nubs 156. The nubs 156 may vary in length from 6 mm to 15 mm. The valleys 153 and peaks 155 may have nubs 516 extending from each and the longer nubs 156 may extend from the peaks 155 and the shorter nubs 156 from the valleys 153 or vice versa. The waved core inner wall 154 may provide for friction for the user as well.

The core 144 may include a core first opening 127 toward the core proximal end 118 and a core second opening 129 toward the core distal end 148. The core 144 may be interchangeable between the core proximal end 118 and core distal end 148 and may be mirror images of each other.

The core 144 may be comprised of two, mirrored pieces of shore 00-30 silicone; however, it will be appreciated that different variations of silicone and polymers are contemplated. The core 18 may be inserted into either a first opening 121 toward the housing proximal end 124 or second opening 123 toward the housing distal end 126.

Referring to FIGS. 16a and 16b, applies to both the first embodiment 10 and second embodiment 110. The first cap 14 is depicted with a face 158, the first opening 15, or aperture, and a first cap ring 162. The face 158 may be outwardly facing in the system 10 (or 110) and may be comprised of material similar to the core 144. The first cap ring 162 may be comprised of material substantially similar to the housing 12 with the face 158 and first cap ring 162 bonded together. The first opening 15 may be an elongated opening extending across a substantially portion of the face 158 without engaging the first cap ring 162. The first opening 15 may resemble human genitalia in its appearance. The more rigid first cap ring 162 may resist flexing and retain itself with a first circumferential groove 166 that may engage the housing proximal end 30 (or 124) first circumferential ring 38 of the housing 12 (or 132 or housing 112). While the softer face 158 of the first cap 14 is the area of direct interface with a user and provides more comfort and pliability. The first cap may be a singular piece of silicone, produced by a dual durometer, two-shot, injection molded liquid silicone rubber (LSR).

The first cap 14 may “snap” onto the housing 12 via the first circumferential ring 132 and the core first ring 62 (or 162) slide or “snap” into the first circumferential groove 166 of the first cap 14. The first cap 14 may include a first lip 68 that may reside within a first circumferential ring groove 67 of the housing 12 (see FIG. 13). The first cap 14 may be held frictionally against the housing as described herein and a frictional force may be applied to release the first cap 14 from the housing 12.

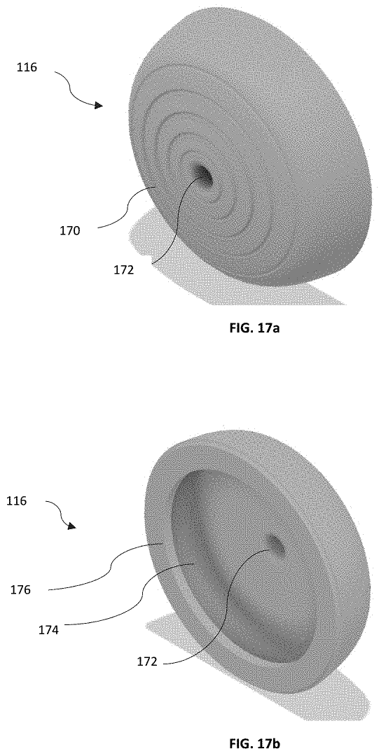

Referring to FIGS. 17a and 17b, a second cap 116 is depicted which may be comprised of a single piece of shore A30 silicone, which may be the same as the housing 112; however, other materials are contemplated herein. The second cap 116 may be shaped substantially similar to first cap 14 and may engage a distal end 126 of the housing 112. The second cap 116 may include a second face 170 and a second aperture 172, which may extend along the central axis when engaged with the housing 112. The second aperture 172, may be a single hole that allows a user to control airflow into and out of the system with a finger or other element that may cover the second aperture 172. Alternatively, the second cap 116 may include no aperture.

The second cap 116 may include a second circumferential groove 174 that may engage the housing distal end 126 and second circumferential ring 134 of the housing 112.

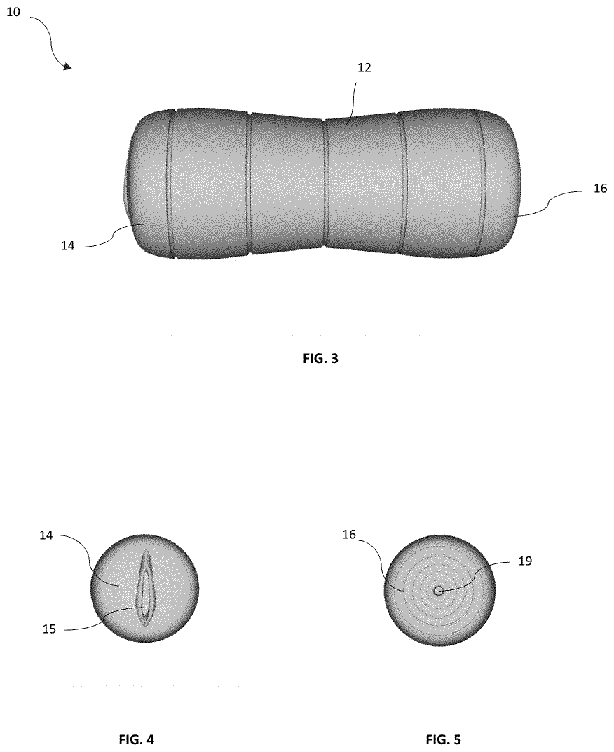

The second cap 116 may “snap” onto the housing 112 via the second circumferential ring 134 and the core second ring 146 slide or “snap” into the second circumferential groove 174 of the second cap 14. The second cap 16 may include a second lip 176 that may reside within a first circumferential ring groove 137 of the housing 12 (refer to FIG. 3). The second cap 16 may be held frictionally against the housing 12 as described herein and a frictional force may be applied to release the second cap 16 from the housing 12.

It will be appreciated that either the first cap 14 or second cap 116 may engage the housing 112 in a plurality of different ways as well including, threading the caps onto the housing, frictional fit without retaining lips (168, 176), or the caps 14, 116 may be irreversibly secured to the housing 112.

The system 110 is intended to be modular and allow a user to easily remove the first cap 14 and second cap 116 as well as the core 44. Each of these elements may be interchangeable or replaced with different designs for the core 44 as well as the first cap 14. The front cap 14 may be provided with different sizes of apertures and different aesthetics to the appearance of genitalia. The second cap 116 may also be interchangeable but may not be changed as frequently. The core 44 may be changed to multiple core designs with different nubs, protrusions, waves, ridges or the like to provide different friction and feedback for a user. The modularity of the system 110 also provides for easy replacement of any component.

The system 110 may be easily manipulated and cleaned by removing one of or both of the first end cap 14 or second end cap 116. The core 144 may then be removed through either one of (or both if the core is multiple parts) the openings of the housing 112. The core 44, housing 112, first end cap 14 and second end cap 116, may be easily cleaned, allowed to dry and then replacing each part as indicated herein.

The system 110 may be manufactured with 100% medical grade platinum-cure silicone which is safe for human skin contact and highly resilient to extreme temperatures, chemicals and mechanical breakdown. Each element of the system 10 may be textured, even subtly textured, to provide extra grip and reduce the probability of lint collection.

Although the foregoing disclosure provides many specifics, these should not be construed as limiting the scope any of the ensuing claims. Other embodiments may be devised which do not depart from the scopes of the claims. Features from different embodiments may be employed separately or in combination. Accordingly, all additions, deletions and modifications to the disclosed subject matter that fall within the scopes of the claims are to be embraced thereby. The scope of each claim is indicated and limited only by its plain language and the full scope of available legal equivalents to its elements.

What is claimed:

1. A modular system comprising:
a silicone sleeve of a first rigidity comprising:
a first semi-cylindrical shell housing;
a second semi-cylindrical shell housing configure to engage the first shell housing;
a fastening mechanism to reversibly engage the first shell housing to the second shell housing;
the first shell housing and second shell housing when engaged comprising:
a cylindrical body and outer wall and an inner wall
a proximal end with a first circumferential ring; and
a silicone core of a second rigidity; and
a first cap positioned toward a proximal end of the silicone sleep, the first cap comprising a first aperture.

2. The modular system of claim 1, wherein the first semi-cylindrical shell housing and second semi-cylindrical shell housing comprise complementary planar surfaces configured to engage each other so as to make the cylindrical body outer wall and inner wall flush.

3. The modular system of claim 1, wherein the silicone core is less rigid than the silicone sleeve.

4. The modular system of claim 1, wherein the silicone core comprises a first portion and a second portion.

5. The modular system of claim 1, wherein the silicone core comprises a first core opening and a second core opening.

6. The modular system of claim 1, wherein the silicone core is positioned within the silicone sleeve and comprises:
a core proximal end with a core first ring, wherein the silicone core extends beyond the proximal end.

7. The modular system of claim 6, wherein the first cap engages both the core first ring and first circumferential ring.

8. A method of assembling and disassembling a collection system comprising:
providing a silicone sleeve with a first rigidity the silicone sleeve comprising:
a cylindrical body and outer wall and an inner wall, the cylindrical body comprising:
a first semi-cylindrical shell housing;
a second semi-cylindrical shell housing configure to engage the first shell housing;
a fastening mechanism to reversibly engage the first shell housing to the second shell housing; and
a proximal end with a first circumferential ring;
placing a silicone core with a second rigidity at least partially within the cylindrical body;
fastening the first semi-cylindrical shell housing to the second semi-cylindrical shell housing;
positioning a first cap over a proximal end of the cylindrical body.

9. The method of claim 8 comprising:
positioning the silicone core entirely within the cylindrical body; and
extending a proximal portion of the silicone core outside the cylindrical body over a circumferential ring of the cylindrical body.

10. The method of claim 8 comprising:
snapping the first cap onto the silicone sleeve.

11. A modular system comprising:
a silicone sleeve of a first rigidity comprising:
a cylindrical body and outer wall and an inner wall
a proximal end with a first circumferential ring; and
a distal end with a second circumferential ring;
a silicone core of a second rigidity;
a first cap positioned toward the proximal end, the first cap comprising a first aperture; and
a second cap positioned toward the distal end, the second cap comprising a second aperture.

12. The modular system of claim 11, wherein the silicone core is less rigid than the silicone sleeve.

13. The modular system of claim 11, wherein the silicone sleeve comprises a first opening and a second opening.

14. The modular system of claim 11, wherein the silicone core comprises a first core opening and a second core opening.

15. The modular system of claim 11, wherein the silicone core is positioned within the silicone sleeve and comprises:
a core proximal end with a core first ring; and
a core distal end with a core second ring;
wherein the silicone core extends beyond both the distal end and the proximal end respectively.

16. The modular system of claim 15, wherein the first cap engages both the core first ring and first circumferential ring.

17. The modular system of claim 15, wherein the second cap engages both the core second ring and the second circumferential ring.

18. A method of assembling and disassembling a collection system comprising:
providing a silicone sleeve with a first rigidity the silicone sleeve comprising:
a cylindrical body and outer wall and an inner wall;
a proximal end with a first circumferential ring; and
a distal end with a second circumferential ring;
sliding a silicone core with a second rigidity at least partially within at least one opening of the silicone sleeve;
extending a core proximal end over the first circumferential ring;
extending a core distal end over the second circumferential ring;
positioning a first cap over the core proximal end and first circumferential ring; and
positioning a second cap over the core distal end and second circumferential ring.

19. The method of claim 18 comprising:
sliding the silicone core entirely through the at least one opening in the silicone sleeve; and
maintaining the silicone core within the cylindrical body of the silicone sleeve.

20. The method of claim 18 comprising:
snapping the first cap onto the silicone sleeve; and
snapping the second cap onto the silicone sleeve opposite the first cap.
